(19) United States
(12) Patent Application Publication
(10) Pub. No.: US 2023/0136754 A1
(43) Pub. Date: May 4, 2023

(54) METHODS AND SYSTEMS OF FACILITATING MANAGEMENT OF MENTAL WELLNESS

(71) Applicants: Daniel Guillermo Asis, Los Reartes (AR); Damián Ariel Klor, Córdoba (AR); Anoop Kumar Kushwaha, Lucknow (IN); Abhay Mohan Wadhwa, New York, NY (US)

(72) Inventors: Daniel Guillermo Asis, Los Reartes (AR); Damián Ariel Klor, Córdoba (AR); Anoop Kumar Kushwaha, Lucknow (IN); Abhay Mohan Wadhwa, New York, NY (US)

(21) Appl. No.: 17/976,897

(22) Filed: Oct. 31, 2022

Related U.S. Application Data
(60) Provisional application No. 63/274,319, filed on Nov. 1, 2021.

(51) Int. Cl. G16H 50/20; G16H 10/20

(52) U.S. Cl. CPC G16H 50/20; G16H 10/20

(57) ABSTRACT

The present disclosure may provide a method of facilitating management of mental wellness. Further, the method may include transmitting a questionnaire information to a user device associated with a user. Further, the questionnaire information is based on a natural language. Further, the method may include receiving a response information from the user device. Further, the response information may comprise a response based on the questionnaire information. Further, the response information is based on the natural language. Further, the method may include analyzing the response information using a first machine learning model. Further, the method may include determining a mental health assessment of the user based on the analyzing of the response information. Further, the method may include identifying a therapy information for the user based on the determination of the mental health assessment of the user.

### CROSS-REFERENCE TO RELATED APPLICATIONS

This application claims the benefit of U.S. Provisional Application No. 63/274,319 filed on 1 Nov. 2021, the contents of which are hereby incorporated by reference.

### FIELD OF DISCLOSURE

The present disclosure generally relates to healthcare informatics, i.e., information and communication technology (ICT) specially adapted for the handling or processing of medical or healthcare data. More specifically, the present disclosure relates to methods and systems of facilitating management of mental wellness.

### BACKGROUND

The field of healthcare informatics is technologically important to several industries, business organizations, and/or individuals.

Existing techniques of facilitating management of mental wellness are deficient in several ways.

Health is a general state of balance that is achieved through many interconnected and integrated variables. The world is becoming urbanized at an unprecedented pace. Globally every day, a sizable set of the human population is migrating to urban areas in search of better opportunities for livelihood and well-being. Living in urban areas, especially large cities have its own advantages but also sometimes takes its toll on not just the physical, but mental well-being of its residents. The ongoing ever-present world condition with technological advancements is having profound mental health consequences for many people around the world. This gives rise to mental health conditions where treatment is required by a large number of people. Most of the urban working population lives under immense stress, mainly because of the competitive work environment, living in nuclear families, lack of sanitation, air pollution, respiratory problems, communicable diseases, traffic jams, accidents, and injuries. People often fail to take out time to relax and rejuvenate as a result they suffer from various lifestyle diseases like—insomnia, hypertension, stress, etc., which may further lead to various physiological and psychological problems including problems related to metabolic activity, anxiety, delirium, and dementia. There are various mental situations where a poor mental health state can result in severe conditions or give an inability to take the right decisions. There are times when individuals are stuck in a crisis situation and become clueless about the next moment, unable to come out of such situations, and ultimately suffer losses on many grounds. An individual's sleeping ability can have a profound impact on his daily routine, regular moods, and body functionality and ultimately, adversely affect his mental state. Such mental state of the individual himself may get addicted to some intoxication due to stress, thus further deteriorating both physical and mental health. Usually, such addictions are not easy to abstain from. Mostly, when individuals are under stress, they usually tend to develop pessimism in them and because of this, they are unable to focus and execute their daily important work. Also, quite often, negative life events and other factors trigger mental states and contribute to adverse psychological states resulting in a combination of symptoms such as constant fear interfering with a person's ability to work, sleep, study, eat, and enjoy interesting activities due to the continuous negative feedback loop. During these covid times, a new disorder called Post Covid Syndrome is intensely impacting the daily lives of people, as it is associated with fatigue ness, weakness, and so on.

It is known that the human brain has innumerable resources to resolve conflicts, overcome sadness, make up for losses, and remain resilient so as to face the hard times that affect us. The possibility of being an active part in the management of one's own health in the effective resolution of problems caused by stress enhances positive aspects of the personality that are related to adaptive stress management.

Previously, there have been multiple attempts made to resolve the above-mentioned problems by employing sensory stimulation through a method implemented by an online platform for providing therapy to individuals. The sensory stimulation generally included stimulation via light, sound, and vibrations at specific wavelengths and frequencies provided via the online platform. The existing technologies are addressing mental health with individual methods, which can be for meditation or reaching a desired mental state like relaxation, or enhanced attention or separate color therapy methods are being provided, but it is seen that generalized and the same treatment are meant to be used on each individual taking the therapy/treatment or the patterns received via the users' input in therapies are not appropriately analyzed. Also, the existing technologies do not suitably assess the user input to provide the user with feedback, suggestions, and notifications to the user for maintaining their mental health. It would be more beneficial when combined advantages of multiple methods and elements are employed in a sequential manner and it may result in a faster and long-lasting solution.

But still, the existing technologies are unable to overcome the existing drawbacks due to a lack of consideration of parameters for assessing and diagnosing the individual's mental health and also due to the lack of collection of appropriate and relevant information at required times as an input from the individuals for useful interpretation of the information. An individual may have more than one mental disorder at a time, and in such cases, existing technologies do not facilitate managing all the mental disorders on a single platform so as enable the person completely to self-manage their mental health.

Therefore, there is a need for methods and systems of facilitating management of mental wellness that may overcome one or more of the above-mentioned problems and/or limitations.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure may provide a method of facilitating management of mental wellness. Further, the method may include transmitting, using a communication device, a questionnaire information to a user device associated with a user. Further, the questionnaire information is based on a natural language. Further, the method may include receiving, using the communication device, a response information from the user device. Further, the response information may comprise a response based on the questionnaire information. Further, the response information may be based on the natural language. Further, the method may include analyzing, using a processing device, the response information using a first machine learning model. Further, the method may include determining, using the processing device, a mental health assessment of the user based on the analyzing of the response information. Further, the method may include identifying, using the processing device, a therapy information for the user based on the determination of the mental health assessment of the user.

The present disclosure may provide a system for facilitating management of mental wellness. Further, the system may include a communication device. Further, the communication device may be configured for transmitting a questionnaire information to a user device associated with a user. Further, the questionnaire information may be based on a natural language. Further, the communication device may be configured for receiving a response information from the user device. Further, the response information may comprise a response based on the questionnaire information. Further, the response information may be based on the natural language. Further, the system may include a processing device. Further, the processing device may be configured for analyzing the response information using a first machine learning model. Further, the processing device may be configured for determining a mental health assessment of the user based on the analyzing of the response information. Further, the processing device may be configured for identifying a therapy information for the user based on the determination of the mental health assessment of the user.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3 illustrates a flowchart of a method 300 of facilitating management of mental wellness, in accordance with some embodiments.

FIG. 4 illustrates a flowchart of a method 400 of facilitating management of mental wellness, in accordance with some embodiments.

FIG. 5 illustrates a flowchart of a method 500 of facilitating management of mental wellness, in accordance with some embodiments.

FIG. 6 illustrates a block diagram of a system 600 of facilitating management of mental wellness, in accordance with some embodiments.

FIG. 7 illustrates a block diagram of the system 600 of facilitating management of mental wellness, in accordance with some embodiments.

FIG. 8 illustrates a block diagram of the system 600 of facilitating management of mental wellness, in accordance with some embodiments.

FIG. 9 illustrates a block diagram of the system 600 of facilitating management of mental wellness, in accordance with some embodiments.

FIG. 10 illustrates a block diagram of the system 600 of facilitating management of mental wellness, in accordance with some embodiments.

FIG. 11 is a block diagram of an architecture 1100 to be used in a system for performing a computer implemented method for diagnosing mental health and providing wellness sessions, in accordance with some embodiments.

FIG. 12 illustrates lists of logic databases L01-L30 to be used in the system for performing the computer implemented method for diagnosing mental health and providing therapeutical/wellness sessions, in accordance with some embodiments.

FIG. 13 illustrates an exemplary questionnaire set 1300 containing questions to appear on the user interface in a particular sequence, in accordance with some embodiments.

FIG. 14 is a block diagram of a system 1400 illustrating the data flow in the system 1400, in accordance with some embodiments.

FIG. 15 is a flow diagram of a method 1500 comprising sequential steps followed by the user for attending a session, in accordance with some embodiments.

FIG. 16 is a flowchart of a method 1600 comprising possible sequential steps for the computer implemented method to be followed depending on the mental state of the individual, in accordance with some embodiments.

FIG. 17 is a flowchart of a computer implemented method 1700 for the condition when the user is unable to identify the exact mental health problems, he/she is undergoing, in accordance with some embodiments.

FIG. 18 is a flowchart of the computer implemented method 1700 for the condition when the user is in a crisis situation, in accordance with some embodiments.

FIG. 19 is a flowchart of the computer implemented method 1700 for the condition when the user is seeking help for daily maintenance of mental health, in accordance with some embodiments.

FIG. 20 is a flowchart of the computer implemented method 1700 for the condition when the user is seeking help for special mental conditions such as addiction, sadness, post-covid syndrome, constant fear, and loss of creativity, in accordance with some embodiments.

FIG. 21 is a flowchart of the computer implemented method 1700 for the condition when the user is seeking help for special mental conditions such as addiction, sadness, post-covid syndrome, constant fear, and loss of creativity, in accordance with some embodiments.

FIG. 22 is a flowchart of a method 2200 for providing mental wellness therapy to individuals, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods and systems for facilitating management of mental wellness, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g., Windows, Mac OS, Unix, Linux, Android, etc.) in order to provide a user interface (e.g., GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g., transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g., initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

Overview:

The present disclosure describes methods and systems of facilitating management of mental wellness. Further, the disclosed system may be configured for providing mental wellness therapy in a self-managed way to individuals. Further, the disclosed method may include a computer-implemented method that utilizes a sequential combination of sound and color therapy infused with digital art for the users to reprocess their emotional conflicts and personal living conditions and further combined and develop into a mobile application software platform which is capable of recognizing user inputs at all stages for mapping their mental health patterns based on the user inputs overtime against their mental state (positive and negative thoughts), which are further analyzed employing a particular set of algorithms to assist & notify users for maintaining their mental health in a Self-Managed way.

Further, the disclosed system may provide a mobile application software platform that provides tools with scientifically proven techniques, easy to use, with self-tests that may allow the user to measure their evolution, evaluate their progress in solving their emotional conflicts, where the users may have substantial insights, thus enabling them solving their problems, with their own inner resources.

Generally, the mobile application software platform aims at any individual who wants to become integrally aware of their health and is willing to be an active and responsible part of reestablishing the lost balance in a self-managed way.

Further, the computer-implemented method may include conducting an interactive therapeutic wellness session for diagnosing and personalized healing in the user. Further, the computer implemented method comprises presenting a questionnaire and receiving responses to the same via a user interface of an electronic device as an input data intending to capture data corresponding to current or already existing emotion of the user, wherein the questionnaire may be designed to intensively and accurately activate specific region of user's cerebral cortex for enabling the user to decipher their emotion and feed the same as the input data into the electronic device, computing the captured input data to identify mental health problems indicative of a psychological state of the user by mapping the received responses enabling to build a personalized healing protocol for the user, directing the user, via the user interface of the electronic device, to first audio-visual space infused with digital arts transitioning temporally as configured according to the personalized healing protocol for receiving an output for a predetermined interval, assessing the psychological state of the user by ascertaining user's emotional characteristics indicative of healing extent, and checking, based on the assessment, if the ascertained emotional characteristics is below its threshold value, the user is directed to second audio-visual space infused with digital arts temporally transitioning as configured according to the personalized healing protocol for another predetermined interval, else recommending to repeat the above steps for intensive healing, wherein said method comprises summarizing the healing experience by demonstrating analysis of the interactive session and showing various results, feedback and user related other information via the user interface.

Further, in some embodiments, the input data may include user responses capturing a severity of the emotion, wherein said output comprises a plurality of healing frequencies and a wavelength configured to elicit a desirable positive response within the user.

Further, in some embodiments, a personalized healing protocol comprises—a. Preparation Stage comprising instructing the user to be prepared for experiencing the personalized healing; b. Treatment Stage comprising preparatory, intermediate, and final phases sequentially and temporally transitioning, each having the audio-visual space infused with digital arts for a predetermined interval; and c. Installation Stage comprising preparatory, intermediate, and a final phase sequentially and temporally transitioning, each having the audio-visual space infused with digital arts different than in the treatment phase for another predetermined interval.

Further, in some embodiments, the disclosed method may include storing data related but not limited to the received responses, assessed psychological state, and assigned personalized healing protocol in a database, wherein the audio-visual space infused with digital arts further comprises a plurality of colors functionally in combination with alternating binaural sounds and geometric shapes and patterns.

Further, in some embodiments, the plurality of colors may be temporally transitioned employing a predefined program to another color in a preparatory, an intermediate, and a final phase of the treatment and installation stage based on the input data.

Further, in some embodiments, the disclosed method may include commencing another round of intensive healing from a preparation stage by formulating another personalized healing protocol as per the predefined program of the method if the ascertained emotional characteristics after a treatment stage are found to be equal to or more than its threshold value. Further, the commencing another round of intensive healing from the preparation stage by formulating another personalized healing protocol as per the predefined program of the method is performed unless the ascertained emotional characteristics after the treatment stage are found to be less than their threshold value.

Further, in some embodiments, the method comprises presenting a set of predefined instructional statements employing the predefined program based on the user input via the user interface for guiding the user through the preparation, treatment, and installation stage, and positively affecting the user's psychological state towards intensive healing.

Further, in some embodiments, said method comprises altering visual characteristics of the user interface depending on the input data employing the predefined program during the interactive session.

Further, in some embodiments, the disclosed system may be a computer-driven system for diagnosing and providing personalized healing in a user via an interactive session comprising a user interface configured to receive input data including user responses via a questionnaire enabling to identify the current or already existing emotion and its severity, a plurality of databases containing a plurality of user stimulating elements configured to intensively and accurately activate a specific region of user's cerebral cortex, a data processing means coupled to the plurality of databases via a communication interface for retrieving and computing input data and accordingly assigning a personalized healing protocol employing the system's predefined program for the user, assessing a psychological state of the user by ascertaining healing extent through the personalized healing protocol, and outputting a comprehensive analysis, summary, rewards, feedback, and takeaways via the user interface.

Further, in some embodiments, the disclosed system may include a data storing means configured to store data from the plurality of databases and the data processing means throughout the interactive session. Further, the stimulating elements may be but are not limited to a plurality of set of statements, sound, color, and digital arts.

Further, in some embodiments, the personalized healing protocol may be designed employing the plurality of user-stimulating elements depending on the user input and the system's program configured to engage the user by exposing them to an audio-visual space infused with digital arts for receiving its output for a predetermined interval.

Further, in some embodiments, the plurality of databases may include a user database containing Full Name, User's Date of birth, color preferences, emotional characteristics, and severity, and a logic database configured to be utilized by the data processing means for assigning and performing the personalized healing protocol, assessing the user's psychological state and instructing via statements during the interactive session.

Further, in some embodiments, the computer-implemented method may employ artificial intelligence applying machine learning, deep learning, and other advanced techniques to solve mental health problems for each individual in a personalized manner. This disclosed method to be performed based on the computer-driven system enables the user to identify their problems, select and monitor a suitable wellness program on a regular basis, and get diagnosed with the treatment on their own in a self-managed manner.

Further, in some embodiments, the present disclosure describes incorporating psycho-analysis with neuro-linguistic programming for diagnosing personal problems and providing therapies through the use of virtual therapy rooms that utilize light, color, and abs (alternating binaural sounds).

Further, the present disclosure describes Pcube-wellness, an exemplary embodiment of the disclosed system herein. Further, the Pcube-wellness is a personal problem processor designed to help solve conflicts, reprocess disturbing memories, expand creativity, and even more. Further, the Pcube wellness is an easy-to-use application that provides tools with scientifically proven techniques and self-test that allow measuring and evaluating a person's progress. The application helps in dealing with psychological conflicts, emotional suffering, stress, addiction, and problems in interpersonal relationships. Pcube-wellness may empower a person to face and overcome their tough moments, find or reestablish lost balance and highlight the positive aspects of their personality. Pcube-wellness is an app that empowers a person (or the user) to face and overcome their tough moments and reestablish lost balance in a self-managed way. The application helps the person in dealing with and overcoming different moments and problems. The application provides a problem processor, that helps the person in identifying specific problems or emotional conflicts, stress, addictions, and relationship problems. Further, the disclosed system may be configured for crisis management in a situation where the person has a short time for receiving the solution, such as nervousness before an interview, a big match, or an event. In this case, the person needs help controlling the situation. Further, the application may facilitate daily maintenance. This is for the person's daily wellness. It is a method aimed at daily maintenance of their positive mental wellness. Further, the application may address special conditions or specific situations that may require continuous development toward achieving a positive goal. The situations included here are
Smoking addiction: A therapy provided by the disclosed system may be ideal for smokers who want to quit.
Sadness/Sad Memories: This therapy was created to overcome sad memories and cope with emotional stress.
Fear and Phobia-related Disorders: If anyone has panic disorder, phobia-related disorders, or there is something that someone is afraid of, then this therapy eases that fear and promotes positivity to combat their fear.
Creative Blocks: When a person is suffering from creative blocks. There can be many reasons for the creative block, including stress and PTSD. This therapy may help the person overcome creative blocks.
Any kind of Post-Covid Fatigue Syndrome related to mental wellness: From the early stages of the pandemic, there have been concerns about its effects on mental health and wellness. The pandemic can be considered a major traumatic event for many people and the symptoms of PCS are similar to PTSD, such as less self-confidence, memory loss, anxiety, or depression. This therapy can help a person overcome it.
Sleep: Stress and sleep are closely related. Almost all people with depression, stress, and anxiety experience sleep issues. This therapy will help a person deal with and overcome sleep issues.
Social Anxiety: If a person suffers from social anxiety or social disorder this therapy will help the person in easing and deal with the fear.

Further, the application may include a condition for seniors, focusing on helping older people maintain their mental wellness and intimacy, concentrating on developing more passionate lovemaking.

Further, the application may provide analysis, progress tracking, and history. Further, the application may help a person in finding guidance toward balance and wellbeing.

Further, the Pcube-wellness is going to be there to help a person in getting guidance and support towards balance and wellbeing.

After completing a session, the person may access their evaluation and track their progress in the reports section. There are three analyses to help a person in their journey: History, Analysis, and Takeaways.

Further, the application may include a proprietary tracking method that shows a person in real-time where the person is in their journey and where the person spends their maximum amount of time. Then through machine learning and basic artificial intelligence, the app is able to provide better guidance in the person's journey over usage and time.

In the History section, the person may be able to see all their activity and procedures in the app. The person may see the history in a graph and select the periods the person wants to see displayed, per day, week, or month. The person may also see their past data analyses via this type of mapping.

In the analysis section, the person can access the before and after treatment conditions side-by-side. This is presented on a map with concentric circles, with the person's positive color in the background.

In the Takeaway section, the person may find the takeaways the person got at the end of each section and may read them again. These are the positive affirmations the person will get, combined with the positive color after each session.

The application analyzes the information to assist and notify the person in maintaining their mental wellness in a self-managed way. Pcube-wellness is going to be with the person on their mental wellness journey. Further, the application may be the person's companion. Further, the Pcube-wellness is for a person at every stage and ready to help and guide the person in their path.

In further embodiments, the computer-implemented method may include conducting an interactive therapeutic wellness session for diagnosing and personalized healing in a user. Further, the computer-implemented method may include presenting a questionnaire and receiving responses to the same via a user interface of an electronic device as an input data intending to capture data corresponding to current or already existing emotion of the user, wherein said questionnaire is designed to intensively and accurately activate a specific region of a user's cerebral cortex for enabling the user to decipher their emotions and feed the same as the input data into the electronic device. Further, the computer-implemented method may include computing the captured input data to identify mental health problems indicative of a psychological state of the user by mapping the received responses enabling to build a personalized healing protocol for the user. Further, the computer-implemented method may include directing the user, via the user interface of the electronic device, to a first audio-visual space infused with digital arts transitioning temporally as configured according to the personalized healing protocol for receiving an output for a predetermined interval. Further, the computer-implemented method may include assessing the psychological state of the user by ascertaining user's emotional characteristics indicative of the healing extent. Further, the computer-implemented method may include checking, based on the assessment, if the ascertained emotional characteristics are below its threshold value, the user is directed to a second audio-visual space infused with the digital arts temporally transitioning as configured according to the personalized healing protocol for another predetermined interval, else recommending to repeat the above steps for intensive healing.

In further embodiments, the computer-driven system may be configured for diagnosing and providing personalized healing in a user via an interactive session. Further, the computer-driven system may include a user interface configured to receive input data including user's responses via a questionnaire enabling to identify the current or already existing emotion and its severity. Further, the computer-driven system may include a plurality of databases containing a plurality of user-stimulating elements configured to intensively and accurately activate a specific region of a user's cerebral cortex. Further, the computer-driven system may include a data processing means coupled to the plurality of databases via a communication interface for retrieving and computing input data and accordingly assigning a personalized healing protocol employing the system's predefined program for the user. Further, the communication interface may be configured for assessing the psychological state of the user by ascertaining the healing extent through the personalized healing protocol. Further, the communication interface may be configured for outputting a comprehensive analysis, summary, rewards, feedback, and takeaways via the user interface.

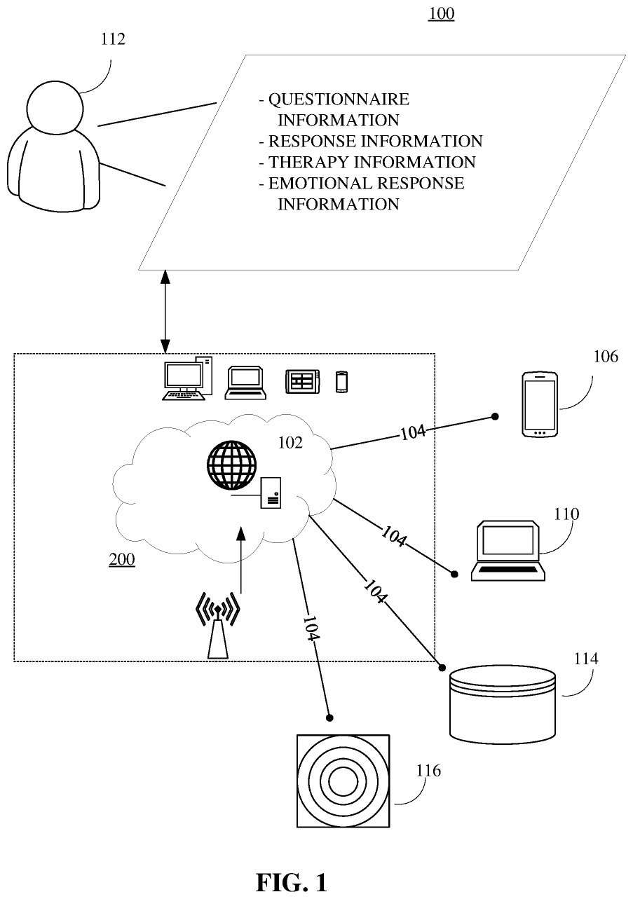

Referring now to figures, FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 200.

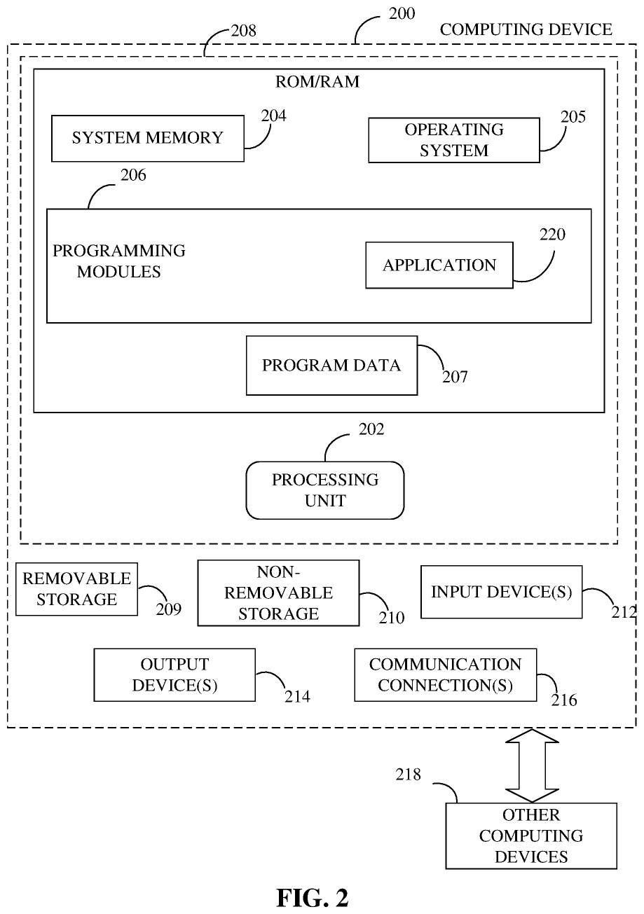

With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g., random-access memory (RAM)), non-volatile (e.g., read-only memory (ROM)), flash memory, or any combination. System memory 204 may include operating system 205, one or more programming modules 206, and may include a program data 207. Operating system 205, for example, may be suitable for controlling computing device 200's operation. In one embodiment, programming modules 206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 2 by those components within a dashed line 208.

Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 204, including operating system 205. While executing on processing unit 202, programming modules 206 (e.g., application 220 such as a media player) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

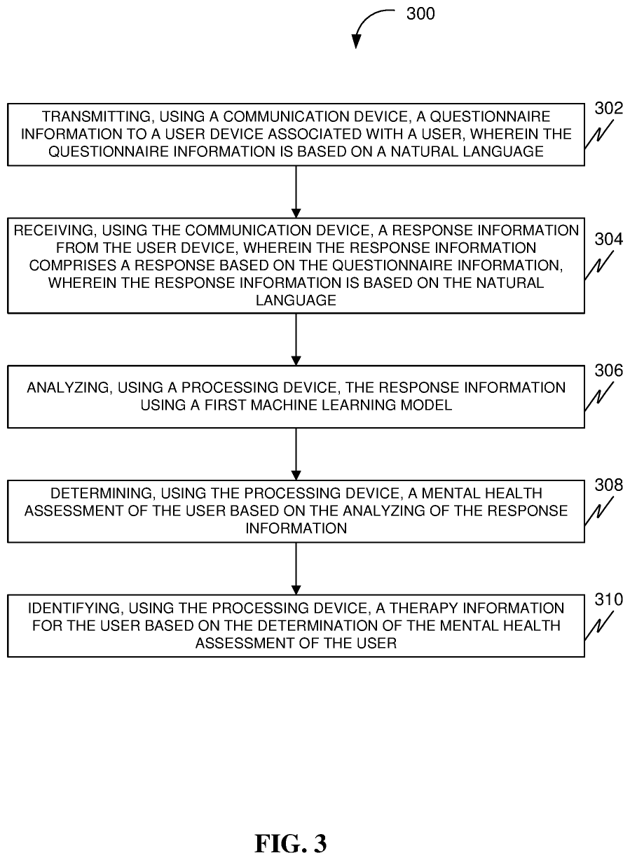

FIG. 3 illustrates a flowchart of a method 300 of facilitating management of mental wellness, in accordance with some embodiments.

Accordingly, the method 300 may include a step 302 of transmitting, using a communication device 602, a questionnaire information to a user device (such as a user device 802) associated with a user. Further, the questionnaire information is based on a natural language. Further, the questionnaire information may include a set of questions for instance, could you tell me about any times over the past few months that you've been bothered by low feelings, stress, or sadness, how frequently have you been bothered by not being able to stop worrying, etc. Further, the method 300 may include a step 304 of receiving, using the communication device 602, a response information from the user device. Further, the response information may include a corresponding answer to its corresponding question present in the questionnaire information. Further, the response information may comprise a response based on the questionnaire information. Further, the response information is based on the natural language. Further, the natural language may include a human language such as English, French, etc. Further, the method 300 may include a step 306 of analyzing, using a processing device 604, the response information using a first machine learning model. Further, the method 300 may include a step 308 of determining, using the processing device 604, a mental health assessment of the user based on the analyzing of the response information. Further, the mental health assessment may include at least one mental health assessment associated with the user. Further, the method 300 may include a step 310 of identifying, using the processing device 604, a therapy information for the user based on the determination of the mental health assessment of the user. Further, the therapy information may include a treatment such as music therapy, patterns and combinations, etc.

In some embodiments, the identifying of the therapy information is performed using a second machine learning model.

In some embodiments, the therapy information may comprise a healing frequency indication based on the therapy information. Further, the healing frequency indication may include a sound light, etc. configured to elicit a desirable positive response within the user. Further, the healing frequency indication may represent a frequency of stimulus which may be configured to elicit the desirable positive response within the user. Further, the method 300 may include transmitting, using the communication device 602, the therapy information to the user device. Further, the user device may comprise a stimulus generator (such as a stimulus generator 1002) which may be configured to generate a stimulus corresponding to the frequency based on the healing frequency indication. Further, the frequency of stimulus may include a sound, light, etc. Further, the desirable positive response may include an emotion of feeling comfortable, an emotion of feeling happy, etc. Further, the stimulus generator may include an electronic device configured to generate the stimulus.

In some embodiments, the method 300 may include generating, using the processing device 604, the questionnaire using a third machine learning model.

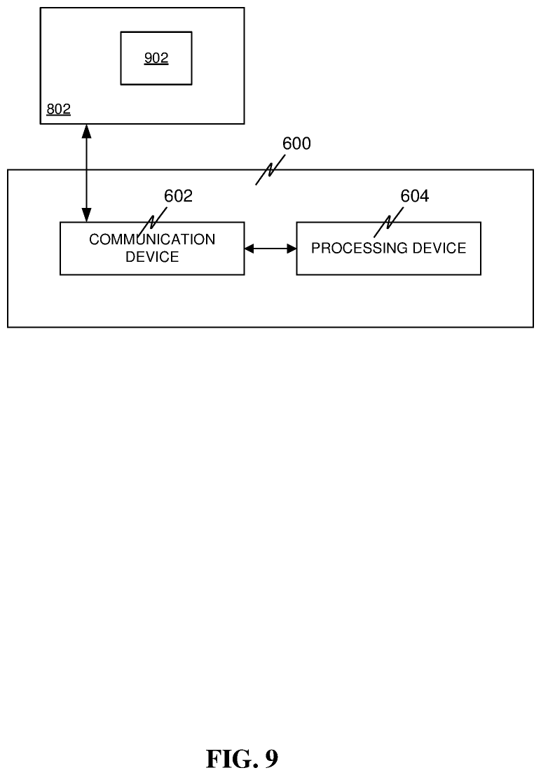

In some embodiments, the method 300 may include transmitting, using the communication device 602, the therapy information to the user device. Further, the therapy information may comprise an audio-visual space indication. Further, the audio-visual space indication may represent an audio-visual content that may be configured to elicit a desirable positive response within the user. Further, the user device may comprise a stimulus generator (such as a stimulus generator 902 (as shown in FIG. 9) which may be configured to generate a stimulus corresponding to the audio-visual content based on the audio-visual space indication. Further, the audio-visual space indication may include an indication for audio-visual content such as a television, an LCD project, a film projector, etc. Further, the stimulus generator may include an electronic device configured to generate the stimulus.
In some embodiments, the questionnaire information may comprise a first questionnaire information and a second questionnaire information. Further, the transmitting of the second questionnaire information may occur subsequent to the transmitting of the first questionnaire information. Further, the first questionnaire information may include a set of questions similar to the questionnaire information. Further, the second questionnaire information may include a set of questions similar to the questionnaire information. Further, the response information may comprise a first response information corresponding to the first questionnaire information and a second response information corresponding to the second questionnaire information. Further, the first response information may include a set of answers associated with the first questionnaire information. Further, the second response information may include a set of answers associated with the second questionnaire information. Further, the mental health assessment may comprise a first mental health assessment corresponding to the analyzing of the first response information and a second mental health assessment corresponding to the analyzing of the second response information. Further, the first mental health assessment may include at least one mental health assessment associated with the user based on the first response information. Further, the second mental health assessment may include at least one mental health assessment associated with the user based on the second response information. Further, the therapy information may comprise a first therapy information corresponding to the first mental health assessment and a second therapy information corresponding to the second mental health assessment. Further, the first response information may comprise an emotional characteristic information representing an emotion of the user based on the first therapy information. Further, the second therapy information may comprise the audio-visual space indication.

In some embodiments, the identifying of the second therapy information may be performed using a fourth machine learning model.

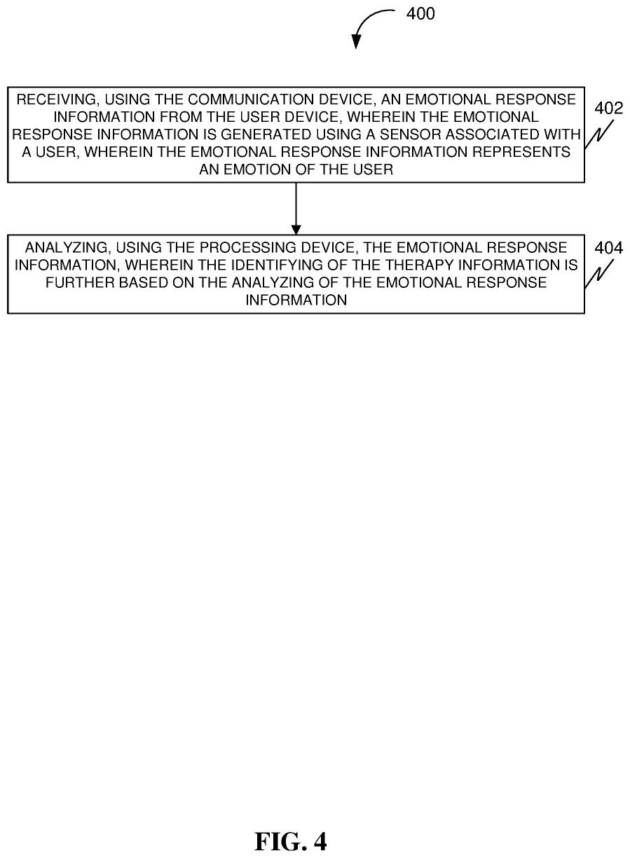

FIG. 4 illustrates a flowchart of a method 400 of facilitating management of mental wellness, in accordance with some embodiments.

Further, in some embodiments, the method 400 may include a step 402 of receiving, using the communication device 602, an emotional response information from the user device. Further, the emotional response information is generated using a sensor associated with the user. Further, the emotional response information may represent an emotion of the user. Further, the emotional response information may include an emotion associated with the user. Further, the sensor may include a computing device such as a camera, smartphone, etc. configured for detecting an emotion associated with the user. Further, the method 400 may include a step 404 of analyzing, using the processing device 604, the emotional response information. Further, the identifying of the therapy information is based on the analyzing of the emotional response information.

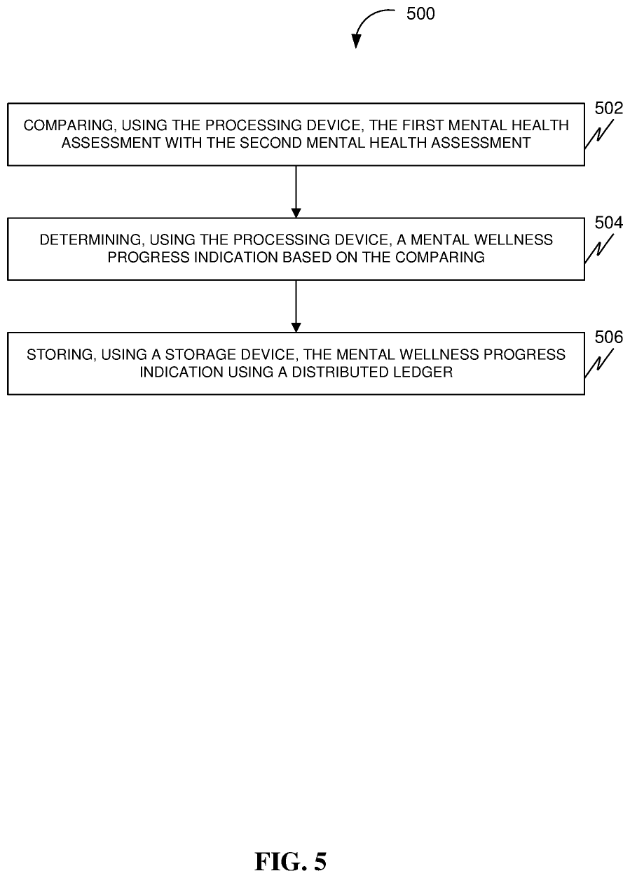

FIG. 5 illustrates a flowchart of a method 500 of facilitating management of mental wellness, in accordance with some embodiments.

Further, the questionnaire information may include a first questionnaire information and a second questionnaire information. Further, the first questionnaire information may include a set of questions similar to the questionnaire information. Further, the second questionnaire information may include a set of questions similar to the questionnaire information. Further, the transmitting of the second questionnaire information may occur subsequent to the transmitting of the first questionnaire information. Further, the response information may include a first response information corresponding to the first questionnaire information and a second response information corresponding to the second questionnaire information. Further, the first response information may include a set of answers associated with the first questionnaire information. Further, the second response information may include a set of answers associated with the second questionnaire information. Further, the mental health assessment may include a first mental health assessment corresponding to the analyzing of the first response information and a second mental health assessment corresponding to the analyzing of the second response information. Further, the first mental health assessment may include at least one mental health assessment associated with the user based on the first response information. Further, the second mental health assessment may include at least one mental health assessment associated with the user based on the second response information. Further, the method 500 may include a step 502 of comparing, using the processing device 604, the first mental health assessment with the second mental health assessment. Further, the method 500 may include a step 504 of determining, using the processing device 604, a mental wellness progress indication based on the comparing. Further, the mental wellness progress indication may include an information for better guidance for managing of mental health wellness. Further, the method 500 may include a step 506 of storing, using a storage device 702, the mental wellness progress indication using a distributed ledger.

In some embodiments, the therapy information may comprise a first therapy information corresponding to the first mental health assessment and a second therapy information corresponding to the second mental health assessment. Further, the identifying of the second therapy information is based on the mental wellness progress indication. Further, the first therapy may include a treatment such as music therapy, patterns and combinations, etc. based on the first mental health assessment. Further, the second therapy may include a treatment such as music therapy, patterns and combinations, etc. based on the second mental health assessment.

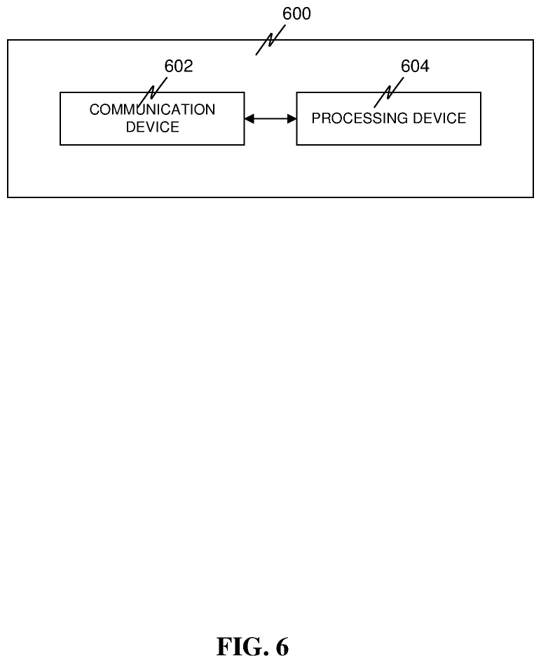

FIG. 6 illustrates a block diagram of a system 600 of facilitating management of mental wellness, in accordance with some embodiments.

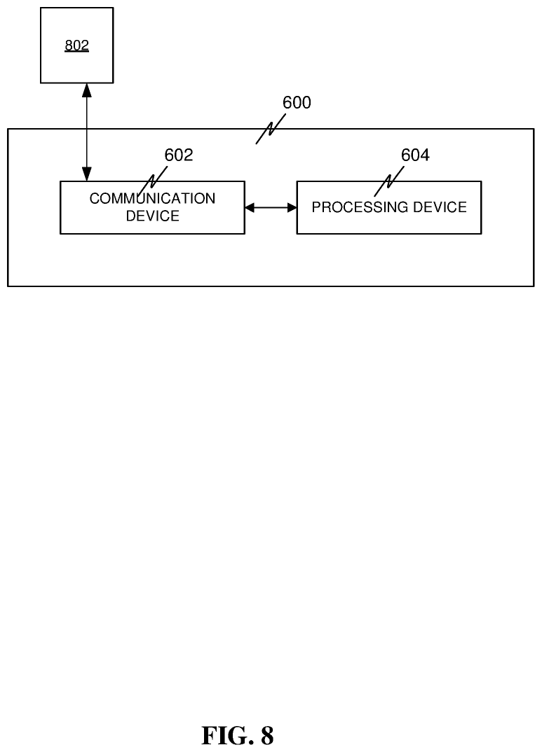

Accordingly, the system 600 may include a communication device 602. Further, the communication device 602 may be configured for transmitting a questionnaire information to a user device 802 (as shown in FIG. 8) associated with a user. Further, the questionnaire information is based on a natural language. Further, the communication device 602 may be configured for receiving a response information from the user device 802. Further, the response information may comprise a response based on the questionnaire information. Further, the response information is based on the natural language. Further, the system 600 may include a processing device 604. Further, the processing device 604 may be configured for analyzing the response information using a first machine learning model. Further, the processing device 604 may be configured for determining a mental health assessment of the user based on the analyzing of the response information. Further, the processing device 604 may be configured for identifying a therapy information for the user based on the determination of the mental health assessment of the user.

In some embodiments, the identifying of the therapy information may be performed using a second machine learning model.

In some embodiments, the communication device 602 further may be configured for receiving an emotional response information from the user device 802. Further, the emotional response information is generated using a sensor associated with the user. Further, the emotional response information may represent an emotion of the user. Further, the processing device 604 may be configured for analyzing the emotional response information. Further, the identifying of the therapy information is based on the analyzing of the emotional response information.

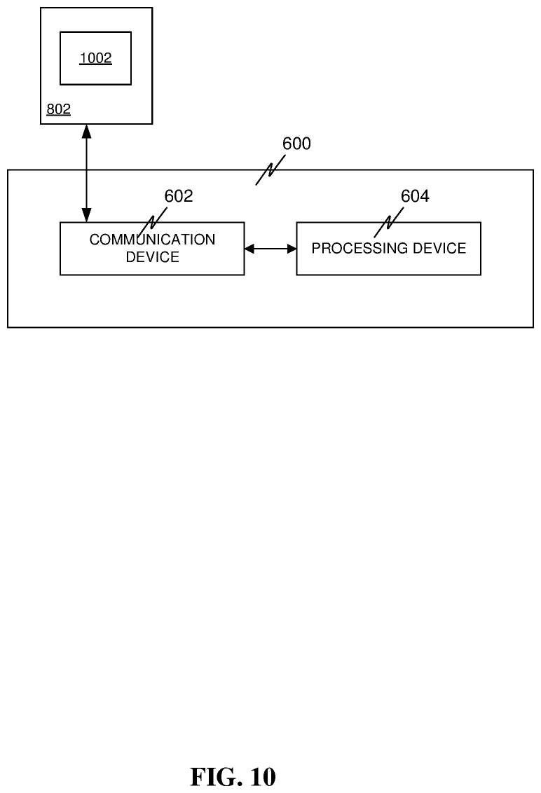

In some embodiments, the therapy information may comprise a healing frequency indication based on the therapy information. Further, the healing frequency indication may represent a frequency of stimulus which may be configured to elicit a desirable positive response within the user. Further, the communication device 602 may be configured for transmitting the therapy information to the user device 802. Further, the user device 802 may comprise a stimulus generator 1002 (as shown in FIG. 10) which may be configured to generate a stimulus corresponding to the frequency based on the healing frequency indication.

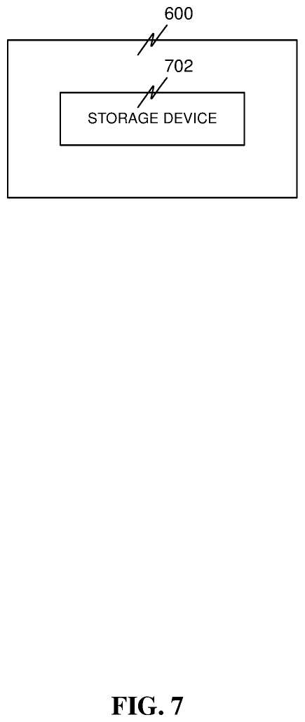

FIG. 7 illustrates a block diagram of the system 600 of facilitating management of mental wellness, in accordance with some embodiments.

Further, in some embodiments, the questionnaire information may include a first questionnaire information and a second questionnaire information. Further, the transmitting of the second questionnaire information may occur subsequent to the transmitting of the first questionnaire information. Further, the response information may include a first response information corresponding to the first questionnaire information and a second response information corresponding to the second questionnaire information. Further, the mental health assessment may include a first mental health assessment corresponding to the analyzing of the first response information and a second mental health assessment corresponding to the analyzing of the second response information. Further, the processing device 604 may be configured for comparing the first mental health assessment with the second mental health assessment. Further, the processing device 604 may be configured for determining a mental wellness progress indication based on the comparing. Further, the system 600 may comprise a storage device 702 (as shown in FIG. 7) which may be configured for storing the mental wellness progress indication using a distributed ledger.

In some embodiments, the therapy information may comprise a first therapy information corresponding to the first mental health assessment and a second therapy information corresponding to the second mental health assessment. Further, the identifying of the second therapy information is based on the mental wellness progress indication.

In some embodiments, the processing device 604 further may be configured for generating the questionnaire using a third machine learning model.

In some embodiments, the therapy information may comprise an audio-visual space indication. Further, the audio-visual space indication may represent an audio-visual content which may be configured to elicit a desirable positive response within the user. Further, the communication device 602 may be configured for transmitting the therapy information to the user device 802. Further, the user device 802 may comprise a stimulus generator 902 (as shown in FIG. 9) which may be configured to generate a stimulus corresponding to the audio-visual content based on the audio-visual space indication.

In some embodiments, the questionnaire information a first questionnaire information and a second questionnaire information. Further, the transmitting of the second questionnaire information may occur subsequent to the transmitting of the first questionnaire information. Further, the response information may comprise a first response information corresponding to the first questionnaire information and a second response information corresponding to the second questionnaire information. Further, the mental health assessment may comprise a first mental health assessment corresponding to the analyzing of the first response information and a second mental health assessment corresponding to the analyzing of the second response information. Further, the therapy information may comprise a first therapy information corresponding to the first mental health assessment and a second therapy information corresponding to the second mental health assessment. Further, the first response information may comprise an emotional characteristic information representing an emotion of the user based on the first therapy information. Further, the second therapy information may comprise the audio-visual space indication.

In some embodiments, the identifying of the second therapy information is performed using a fourth machine learning model.

FIG. 8 illustrates a block diagram of the system 600 of facilitating management of mental wellness, in accordance with some embodiments.

FIG. 9 illustrates a block diagram of the system 600 of facilitating management of mental wellness, in accordance with some embodiments.

FIG. 10 illustrates a block diagram of the system 600 of facilitating management of mental wellness, in accordance with some embodiments.

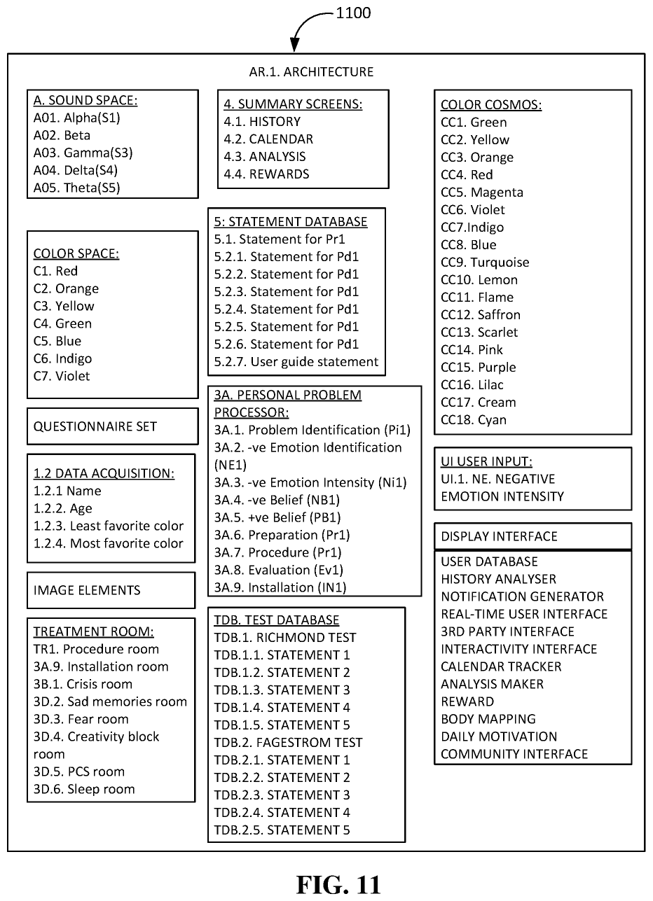

FIG. 11 is a block diagram of an architecture 1100 to be used in a system for performing a computer implemented method for diagnosing mental health and providing wellness sessions, in accordance with some embodiments.

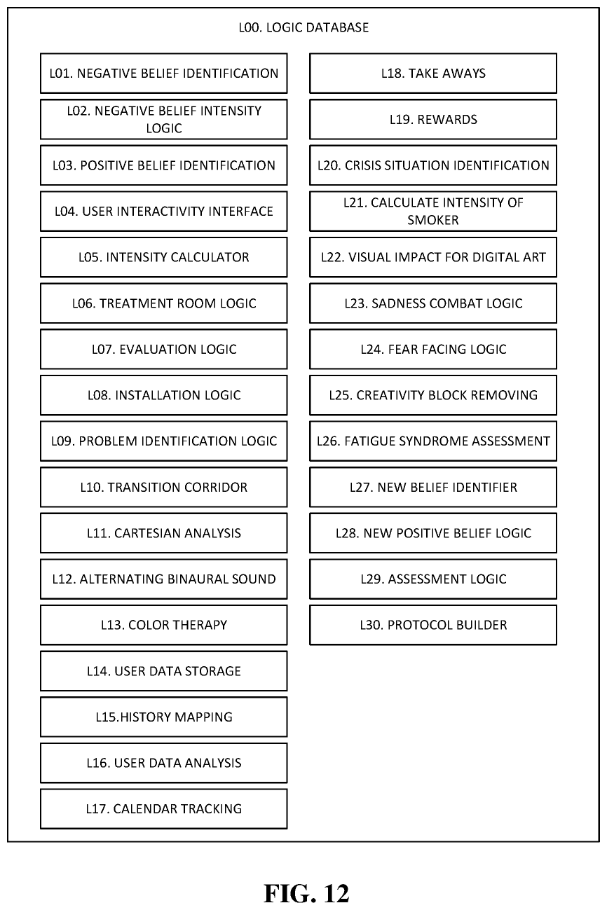

FIG. 12 illustrates lists of logic databases L01-L30 to be used in the system for performing the computer implemented method for diagnosing mental health and providing therapeutical/wellness sessions, in accordance with some embodiments.

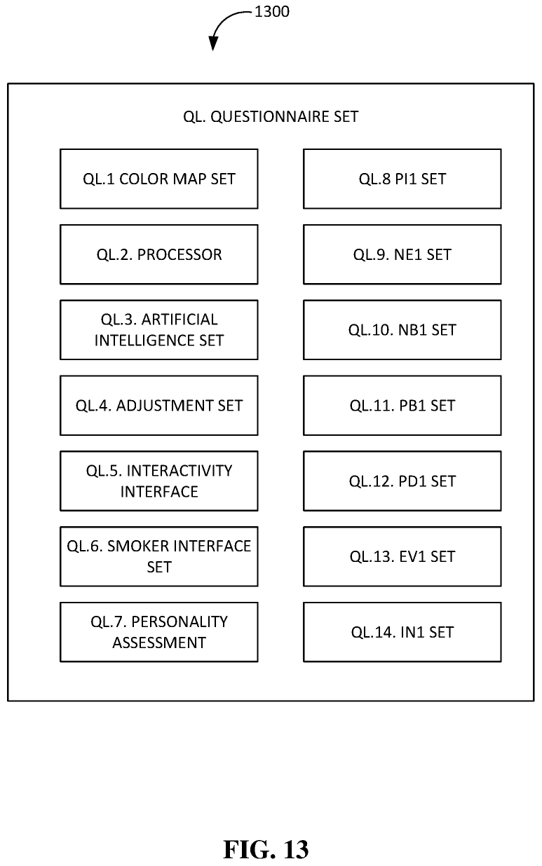

FIG. 13 illustrates an exemplary questionnaire set 1300 containing questions to appear on the user interface in a particular sequence, in accordance with some embodiments.

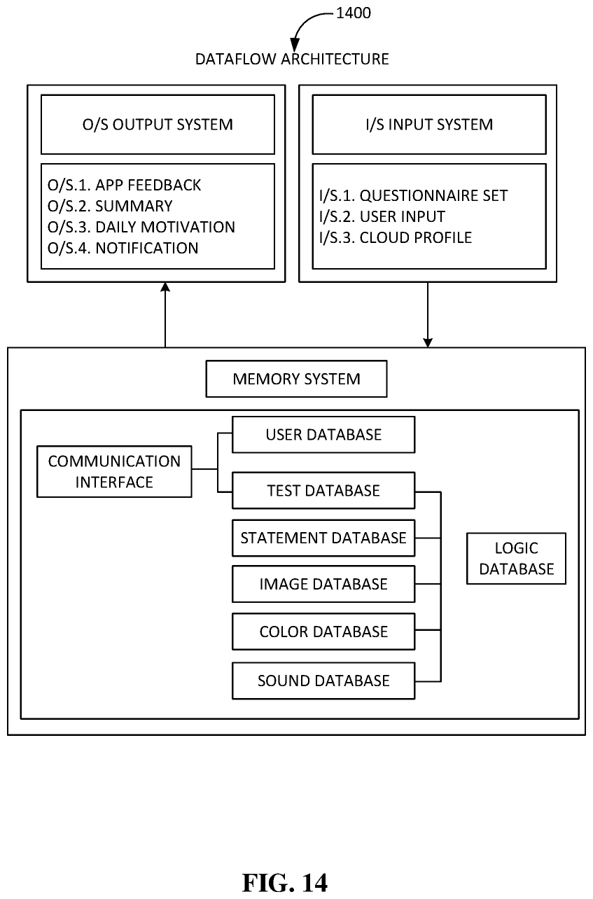

FIG. 14 is a block diagram of a system 1400 illustrating the data flow in the system 1400, in accordance with some embodiments.

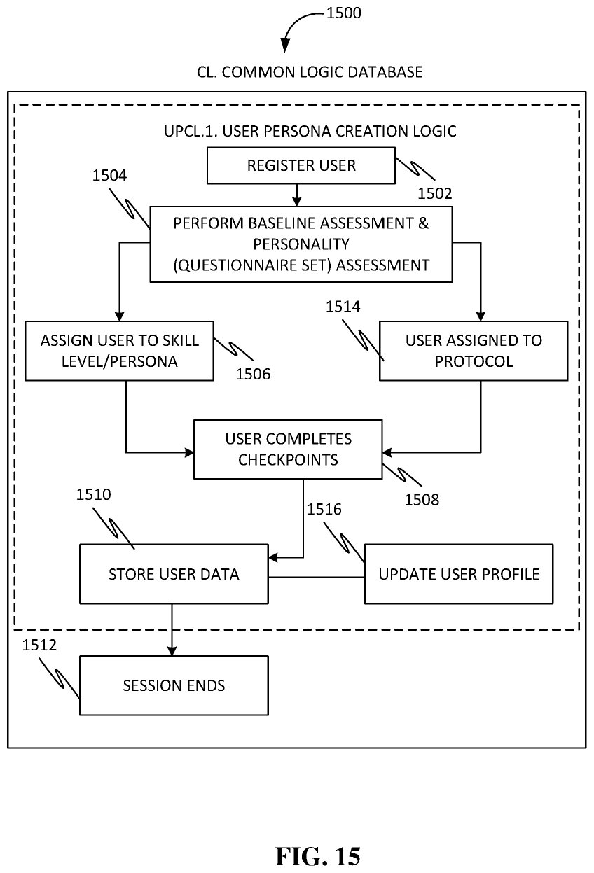

FIG. 15 is a flow diagram of a method 1500 comprising sequential steps followed by the user for attending a session, in accordance with some embodiments. Accordingly, at 1502, the method 1500 may include the user registering himself. Further, at 1504, the method 1500 may include performing a baseline assessment and personality (questionnaire set) assessment. Further, at 1506, the method 1500 may include assigning the user to skill level/persona. Further, at 1508, the method 1500 may include the user completing checkpoints. Further, at 1510, the method 1500 may include storing a user data. Further, at 1512, the method 1500 may include ending the session. Further, after 1504, at 1514, the method 1500 may include assigning the user to a protocol. Further, after 1514, the method 1500 may proceed to 1508. Further, after 1510, at 1516, the method 1500 may include updating a user profile.

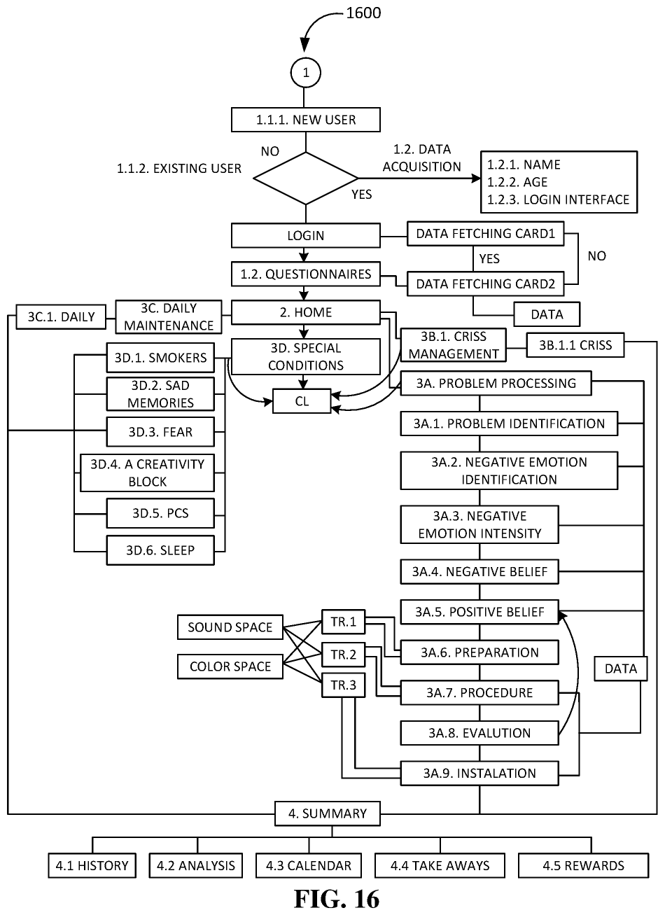

FIG. 16 is a flowchart of a method 1600 comprising possible sequential steps for the computer implemented method to be followed depending on the mental state of the individual, in accordance with some embodiments.

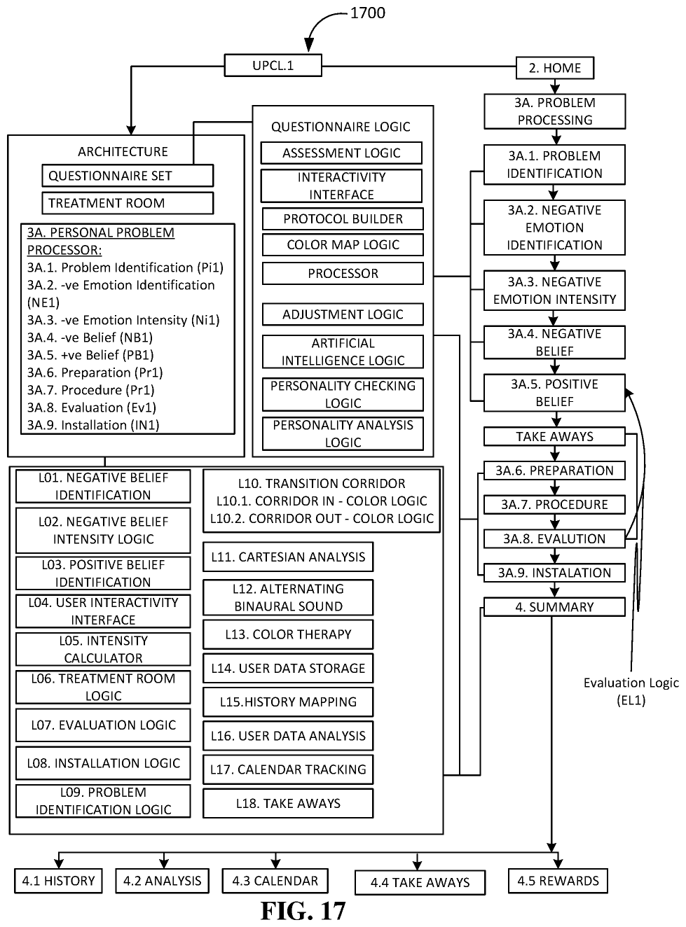

FIG. 17 is a flowchart of a computer implemented method 1700 for the condition when the user is unable to identify the exact mental health problems, he/she is undergoing, in accordance with some embodiments.

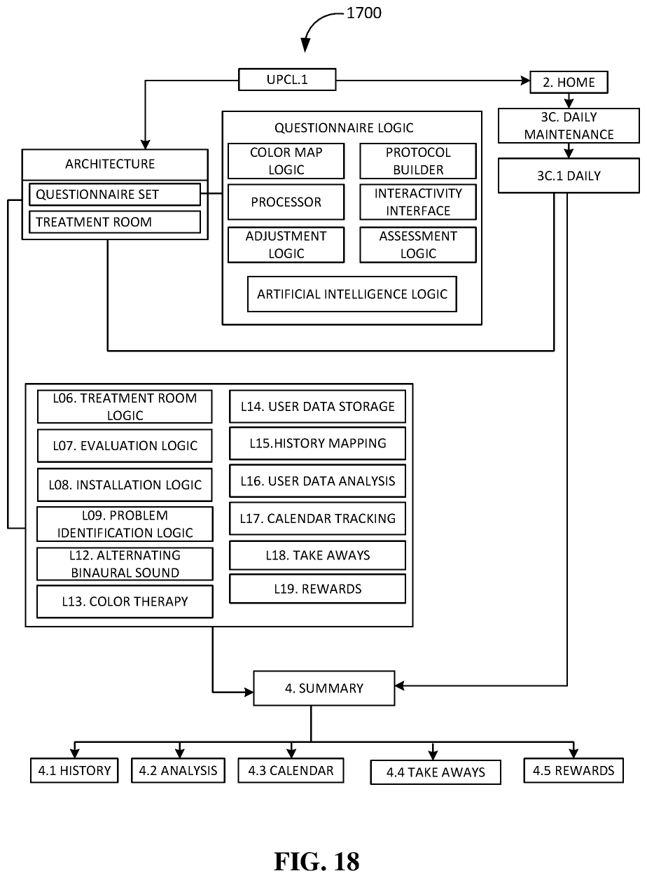

FIG. 18 is a flowchart of the computer implemented method 1700 for the condition when the user is in a crisis situation, in accordance with some embodiments.

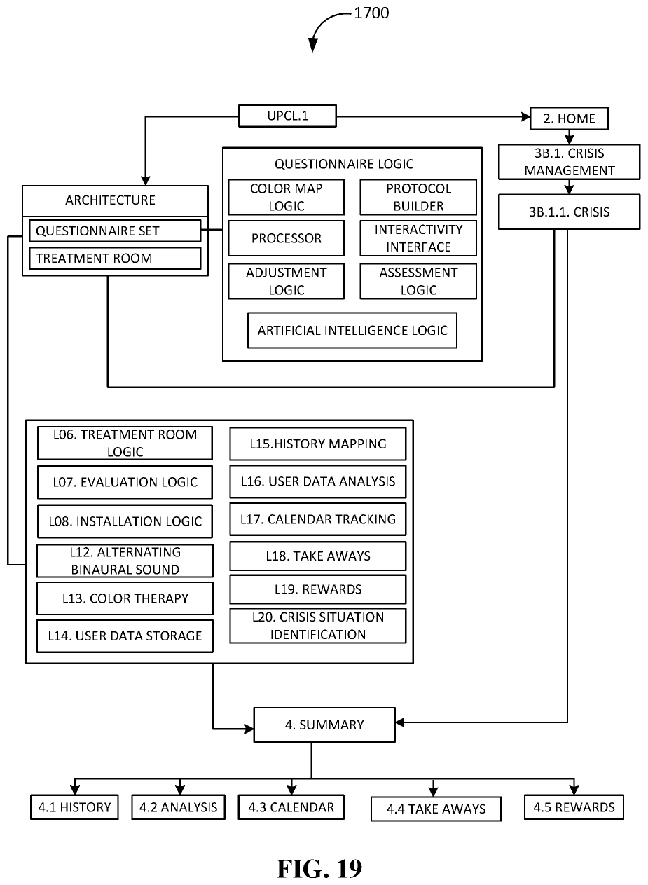

FIG. 19 is a flowchart of the computer implemented method 1700 for the condition when the user is seeking help for daily maintenance of mental health, in accordance with some embodiments.

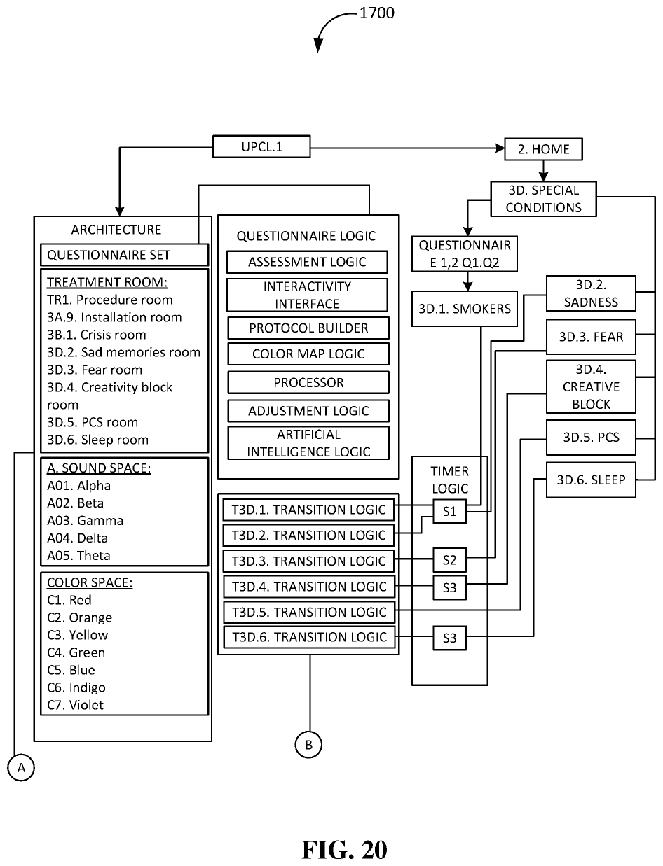

FIG. 20 is a flowchart of the computer implemented method 1700 for the condition when the user is seeking help for special mental conditions such as addiction, sadness, post-covid syndrome, constant fear, and loss of creativity, in accordance with some embodiments.

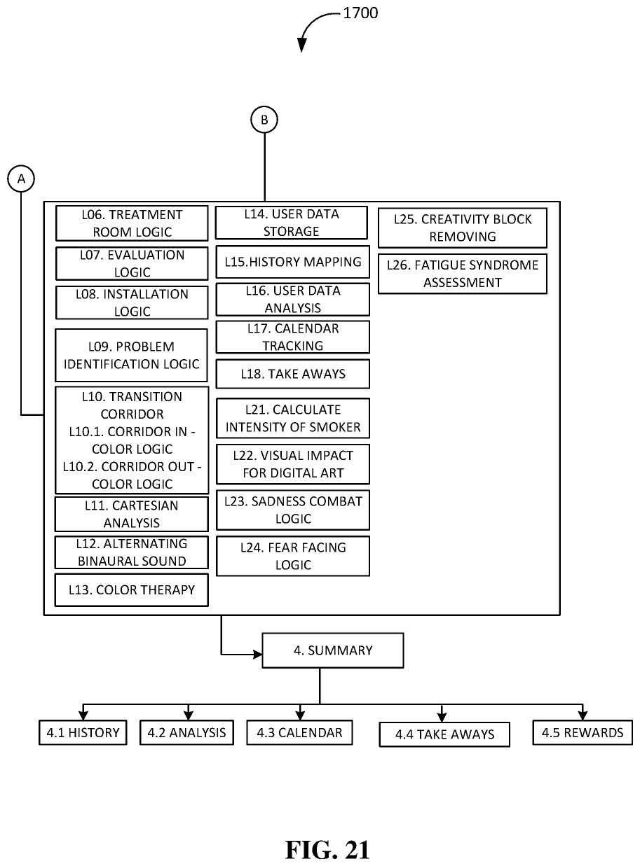

FIG. 21 is a flowchart of the computer implemented method 1700 for the condition when the user is seeking help for special mental conditions such as addiction, sadness, post-covid syndrome, constant fear, and loss of creativity, in accordance with some embodiments.

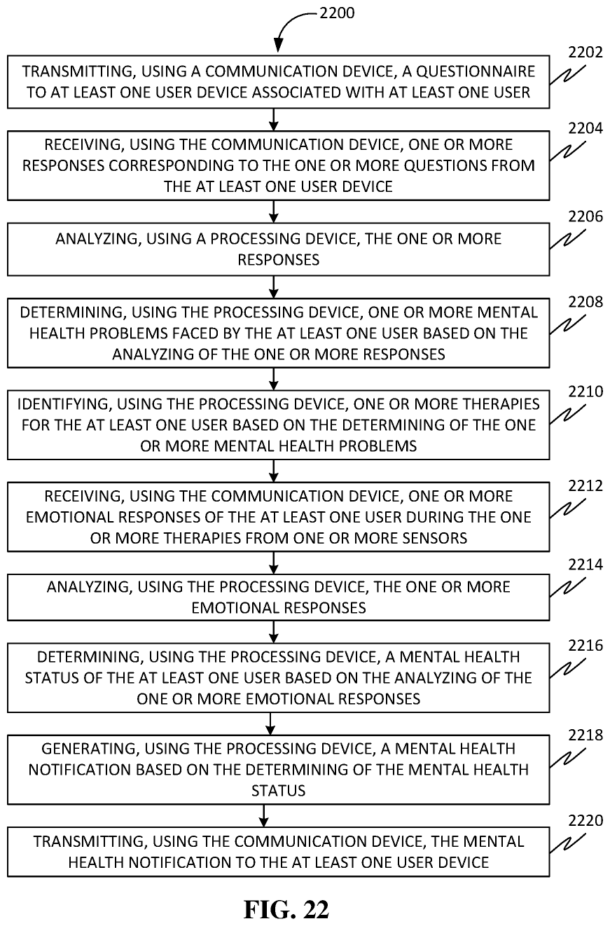

FIG. 22 is a flowchart of a method 2200 for providing mental wellness therapy to individuals, in accordance with some embodiments. Accordingly, at 2202, the method 2200 may include a step of transmitting, using a communication device (such as the communication device 602), a questionnaire to at least one user device (such as the user device 802) associated with at least one user. Further, the questionnaire may include one or more questions.

Further, at 2204, the method 2200 may include a step of receiving, using the communication device, one or more responses corresponding to the one or more questions from the at least one user device.

Further, at 2206, the method 2200 may include a step of analyzing, using a processing device (such as the processing device 604), the one or more responses.

Further, at 2208, the method 2200 may include a step of determining, using the processing device, one or more mental health problems faced by the at least one user based on the analyzing of the one or more responses.

Further, at 2210, the method 2200 may include a step of identifying, using the processing device, one or more therapies for the at least one user based on the determining of the one or more mental health problems. Further, the one or more therapies may be provided to the at least one user.

Further, at 2212, the method 2200 may include a step of receiving, using the communication device, one or more emotional responses of the at least one user during the one or more therapies from one or more sensors. Further, the one or more sensors may be configured for generating the one or more emotional response based on detecting one or more emotional characteristics of the at least one user.

Further, at 2214, the method 2200 may include a step of analyzing, using the processing device, the one or more emotional responses.

Further, at 2216, the method 2200 may include a step of determining, using the processing device, a mental health status of the at least one user based on the analyzing of the one or more emotional responses.

Further, at 2218, the method 2200 may include a step of generating, using the processing device, a mental health notification based on the determining of the mental health status.

Further, at 2220, the method 2200 may include a step of transmitting, using the communication device, the mental health notification to the at least one user device.

Although the invention has been explained in relation to its preferred embodiment, it is to be understood that many other possible modifications and variations can be made without departing from the spirit and scope of the invention as hereinafter claimed.

We claim:

1. A method of facilitating management of mental wellness, the method comprising:
transmitting, using a communication device, a questionnaire information to a user device associated with a user, wherein the questionnaire information is based on a natural language;
receiving, using the communication device, a response information from the user device, wherein the response information comprises a response based on the questionnaire information, wherein the response information is based on the natural language;
analyzing, using a processing device, the response information using a first machine learning model;
determining, using the processing device, a mental health assessment of the user based on the analyzing of the response information; and
identifying, using the processing device, a therapy information for the user based on the determination of the mental health assessment of the user.

2. The method of claim 1, wherein the identifying of the therapy information is performed using a second machine learning model.

3. The method of claim 1, wherein the method further comprises:
receiving, using the communication device, an emotional response information from the user device, wherein the emotional response information is generated using a sensor associated with the user, wherein the emotional response information represents an emotion of the user; and
analyzing, using the processing device, the emotional response information, wherein the identifying of the therapy information is further based on the analyzing of the emotional response information.

4. The method of claim 1, wherein the therapy information comprises a healing frequency indication based on the therapy information, wherein the healing frequency indication represents a frequency of stimulus configured to elicit a desirable positive response within the user, wherein the method further comprises transmitting, using the communication device, the therapy information to the user device, wherein the user device comprises a stimulus generator configured to generate a stimulus corresponding to the frequency based on the healing frequency indication.

5. The method of claim 1, wherein the questionnaire information comprises a first questionnaire information and a second questionnaire information, wherein the transmitting of the second questionnaire information occurs subsequent to the transmitting of the first questionnaire information, wherein the response information comprises a first response information corresponding to the first questionnaire information and a second response information corresponding to the second questionnaire information, wherein the mental health assessment comprises a first mental health assessment corresponding to the analyzing of the first response information and a second mental health assessment corresponding to the analyzing of the second response information, wherein the method further comprising:
comparing, using the processing device, the first mental health assessment with the second mental health assessment; and
determining, using the processing device, a mental wellness progress indication based on the comparing; and
storing, using a storage device, the mental wellness progress indication using a distributed ledger.

6. The method of claim 5, wherein the therapy information comprises a first therapy information corresponding to the first mental health assessment and a second therapy information corresponding to the second mental health assessment, wherein the identifying of the second therapy information is based on the mental wellness progress indication.

7. The method of claim 1, wherein the method further comprises generating, using the processing device, the questionnaire using a third machine learning model.

8. The method of claim 1 further comprising transmitting, using the communication device, the therapy information to the user device, wherein the therapy information comprises an audio-visual space indication, wherein the audio-visual space indication represents an audio-visual content configured to elicit a desirable positive response within the user, wherein the user device comprises a stimulus generator configured to generate a stimulus corresponding to the audio-visual content based on the audio-visual space indication.

9. The method of claim 8, wherein the questionnaire information comprises a first questionnaire information and a second questionnaire information, wherein the transmitting of the second questionnaire information occurs subsequent to the transmitting of the first questionnaire information, wherein the response information comprises a first response information corresponding to the first questionnaire information and a second response information corresponding to the second questionnaire information, wherein the mental health assessment comprises a first mental health assessment corresponding to the analyzing of the first response information and a second mental health assessment corresponding to the analyzing of the second response information, wherein the therapy information comprises a first therapy information corresponding to the first mental health assessment and a second therapy information corresponding to the second mental health assessment, wherein the first response information comprises an emotional characteristic information representing an emotion of the user based on the first therapy information, wherein the second therapy information comprises the audio-visual space indication.

10. The method of claim 9, wherein the identifying of the second therapy information is performed using a fourth machine learning model.

11. A system for facilitating management of mental wellness, wherein the system comprises:
a communication device configured for:
transmitting a questionnaire information to a user device associated with a user, wherein the questionnaire information is based on a natural language; and
receiving a response information from the user device, wherein the response information comprises a response based on the questionnaire information, wherein the response information is based on the natural language; and
a processing device configured for:
analyzing the response information using a first machine learning model;
determining a mental health assessment of the user based on the analyzing of the response information; and
identifying a therapy information for the user based on the determination of the mental health assessment of the user.

12. The system of claim 11, wherein the identifying of the therapy information is performed using a second machine learning model.

13. The system of claim 11, wherein the communication device is further configured for receiving an emotional response information from the user device, wherein the emotional response information is generated using a sensor associated with the user, wherein the emotional response information represents an emotion of the user, wherein the processing device is further configured for analyzing the emotional response information, wherein the identifying of the therapy information is further based on the analyzing of the emotional response information.

14. The system of claim 11, wherein the therapy information comprises a healing frequency indication based on the therapy information, wherein the healing frequency indication represents a frequency of stimulus configured to elicit a desirable positive response within the user, wherein the communication device is further configured for transmitting the therapy information to the user device, wherein the user device comprises a stimulus generator configured to generate a stimulus corresponding to the frequency based on the healing frequency indication.

15. The system of claim 11, wherein the questionnaire information comprises a first questionnaire information and a second questionnaire information, wherein the transmitting of the second questionnaire information occurs subsequent to the transmitting of the first questionnaire information, wherein the response information comprises a first response information corresponding to the first questionnaire information and a second response information corresponding to the second questionnaire information, wherein the mental health assessment comprises a first mental health assessment corresponding to the analyzing of the first response information and a second mental health assessment corresponding to the analyzing of the second response information, wherein the processing device is further configured for:
comparing the first mental health assessment with the second mental health assessment; and
determining, a mental wellness progress indication based on the comparing, wherein the system further comprises a storage device configured for storing the mental wellness progress indication using a distributed ledger.

16. The system of claim 15, wherein the therapy information comprises a first therapy information corresponding to the first mental health assessment and a second therapy information corresponding to the second mental health assessment, wherein the identifying of the second therapy information is based on the mental wellness progress indication.

17. The system of claim 11, wherein the processing device is further configured for generating the questionnaire using a third machine learning model.

18. The system of claim 11, wherein the therapy information comprises an audio-visual space indication, wherein the audio-visual space indication represents an audio-visual content configured to elicit a desirable positive response within the user, wherein the communication device is further configured for transmitting the therapy information to the user device, wherein the user device comprises a stimulus generator configured to generate a stimulus corresponding to the audio-visual content based on the audio-visual space indication.

19. The system of claim 18, wherein the questionnaire information a first questionnaire information and a second questionnaire information, wherein the transmitting of the second questionnaire information occurs subsequent to the transmitting of the first questionnaire information, wherein the response information comprises a first response information corresponding to the first questionnaire information and a second response information corresponding to the second questionnaire information, wherein the mental health assessment comprises a first mental health assessment corresponding to the analyzing of the first response information and a second mental health assessment corresponding to the analyzing of the second response information, wherein the therapy information comprises a first therapy information corresponding to the first mental health assessment and a second therapy information corresponding to the second mental health assessment, wherein the first response information comprises an emotional characteristic information representing an emotion of the user based on the first therapy information, wherein the second therapy information comprises the audio-visual space indication.

20. The system of claim 19, wherein the identifying of the second therapy information is performed using a fourth machine learning model.
